(19) United States
(12) Patent Application Publication
(10) Pub. No.: US 2024/0122929 A1
(43) Pub. Date: Apr. 18, 2024

(54) BODY TEMPERATURE LOWERING AGENT, BODY TEMPERATURE ELEVATION SUPPRESSOR, FOOD COMPOSITION FOR LOWERING BODY TEMPERATURE,AND FOOD COMPOSITION FOR SUPPRESSING BODY TEMPERATURE ELEVATION

(71) Applicant: University of Tsukuba, Ibaraki (JP)

(72) Inventors: Hideaki SOYA, Tsukuba-shi (JP); Takuya SHIBASAKI, Tsukuba-shi (JP)

(21) Appl. No.: 18/276,762

(22) PCT Filed: Feb. 14, 2022
(86) PCT No.: PCT/JP2022/005738

§ 371 Date: Aug. 10, 2023

(30) Foreign Application Priority Data
Feb. 15, 2021 (JP) 2021-021815

(51) Int. Cl. A61K 31/513; A61P 29/02

(52) U.S. Cl. CPC A61K 31/513; A61P 29/02

(57) ABSTRACT

A body temperature lowering agent, a body temperature elevation suppressor, a food composition for lowering body temperature, and a food composition for suppressing body temperature elevation containing at least one substance selected from the group consisting of orotic acid, a derivative thereof, and a salt of the orotic acid or the derivative as an active ingredient.

### TECHNICAL FIELD

The present invention relates to a body temperature lowering agent, a body temperature elevation suppressor, a food composition for lowering body temperature, and a food composition for suppressing body temperature elevation.

The present application claims priority based on Japanese Patent Application No. 2021-21815 filed in Japan on Feb. 15, 2021, the contents of which are incorporated herein by reference.

### BACKGROUND ART

Excessive body temperature elevation increases the risk of heatstroke. For the purpose of preventing this, body temperature elevation has been suppressed by using an ice slurry beverage, wearing of an ice vest, body cooling before exercise (precooling), or the like, but the effect thereof has been limited. One reason for this is that while the physical cooling effect from the outside of the body reaches a portion close to the body surface, heat generation continues in the body, and thus core body temperature elevation cannot be suppressed.

Orotic acid is a precursor of pyrimidine nucleotide synthesis and is contained in dairy products such as cow's milk. Orotic acid is hardly soluble in water, but there is known a production method for utilizing it as a beverage in which precipitation hardly occurs (Patent Document 1 and the like). Orotic acid is also used in Class 2 drugs, Class 3 drugs, quasi-drugs, health foods, and the like for the purpose of nutritional fortification, hepatic function facilitation, and cosmetic effects.

Orotic acid is known to have a myocardial protection action (Non-Patent Document 1), a uric acid level reducing action (Patent Document 2), a reducing action of oxygen consumption and energy consumption (Patent Document 3), an endurance improving action (Patent Document 4), and the like. With regard to the relationship between orotic acid and body temperature, Non-Patent Document 2 reports that the body temperature was not lowered even when orotic acid was administered to a rat, and it is not known that orotic acid lowers body temperature in mammals.

### CITATION LIST

### Patent Documents

Patent Document 1: WO 2015/029368
Patent Document 2: JP 2011-98896 A
Patent Document 3: JP 2012-246280 A
Patent Document 4: JP 2011-136907 A

### Non-Patent Document

Non-Patent Document 1: M. Loffler, E. A. Carrey, E. Zameitat, Journal of Genetics and Genomics, Volume 42, Issue 5, 20 May 2015 Pages 207-219
Non-Patent Document 2: T. A. Bal'magiya, G. A. Antonova & N. A. Khodorova, Bulletin of Experimental Biology and Medicine, volume 79(3), pages 238-240, 1975

### SUMMARY OF INVENTION

### Technical Problem

There has been a problem that external peripheral cooling such as wearing of an ice vest, precooling, and an ice slurry beverage, which are known in the related art as methods for suppressing high body temperature, requires a dedicated instrument, the wearing of the instrument disturbs activities, and a large amount of water is required, but the cooling effect neither reaches the core body nor lasts.

An object of the present invention is to provide a body temperature lowering agent, a body temperature elevation suppressor, a food composition for lowering body temperature, and a food composition for suppressing body temperature elevation, which can lower body temperature spontaneously and continuously and suppress excessive body temperature elevation without requiring an instrument.

### Solution to Problem

The present inventors have found that orotic acid has an effect of lowering core body temperature at rest and suppressing excessive body temperature elevation during exercise, and is useful as a body temperature lowering agent, a body temperature elevation suppressor, a food composition for lowering body temperature, and a food composition for suppressing body temperature elevation, and thus completing present invention.

That is, the present invention includes the following aspects.

[1] A body temperature lowering agent containing at least one substance selected from the group consisting of orotic acid, a derivative thereof, and a salt of the orotic acid or the derivative as an active ingredient.

[2] The body temperature lowering agent according to [1], wherein the body temperature is core body temperature.

[3] The body temperature lowering agent according to [1] or [2], wherein the orotic acid is a free form of orotic acid.

[4] A body temperature elevation suppressor containing at least one substance selected from the group consisting of orotic acid, a derivative thereof, and a salt of the orotic acid or the derivative as an active ingredient.

[5] The body temperature elevation suppressor according to [4], wherein the body temperature is core body temperature.

[6] The body temperature elevation suppressor according to [4] or [5], wherein the orotic acid is a free form of orotic acid.

[7] A food composition for lowering body temperature, containing at least one substance selected from the group consisting of orotic acid, a derivative thereof, and a salt of the orotic acid or the derivative as an active ingredient.

[8] A food composition for suppressing body temperature elevation, containing at least one substance selected from the group consisting of orotic acid, a derivative thereof, and a salt of the orotic acid or the derivative as an active ingredient.

[9] The food composition according to [7] or [8], wherein the body temperature is core body temperature.

[10] The food composition according to any one of [7] to [9], wherein the orotic acid is a free form of orotic acid.

### Advantageous Effects of Invention

According to the present invention, it is possible to provide a body temperature lowering agent, a body temperature elevation suppressor, a food composition for lowering body temperature, and a food composition for suppressing body temperature elevation, which can lower body temperature spontaneously and continuously and suppressing excessive body temperature elevation without requiring an instrument.

### BRIEF DESCRIPTION OF DRAWINGS

FIG. 1 shows transition of core body temperature at rest in a vehicle administration group, a 30 mg/kg orotic acid administration group, and a 300 mg/kg orotic acid administration group. In the figure, “***” indicates that a p-value of the 300 mg/kg orotic acid administration group is less than 0.1% with respect to the vehicle administration group, “$$” indicates that the p-value of the 300 mg/kg orotic acid administration group is less than 1% with respect to the 30 mg/kg orotic acid administration group, and “$$$” indicates that the p-value of the 300 mg/kg orotic acid administration group is less than 0.1% with respect to the 30 mg/kg orotic acid administration group.

FIG. 2 shows transition of a heart rate at rest in the vehicle administration group, the 30 mg/kg orotic acid administration group, and the 300 mg/kg orotic acid administration group.

FIG. 3 shows transition of a heat production amount at rest in the vehicle administration group, the 30 mg/kg orotic acid administration group, and the 300 mg/kg orotic acid administration group. In the figure, “*” indicates that the p-value of the 300 mg/kg orotic acid administration group is less than 5% with respect to the vehicle administration group.

FIG. 4 shows a spontaneous activity amount at rest in the vehicle administration group, the 30 mg/kg orotic acid administration group, and the 300 mg/kg orotic acid administration group.

FIG. 5 shows transition of core body temperature during and after exercise in the vehicle administration group and the 300 mg/kg orotic acid administration group. In the figure, “Exercise” indicates the time (minutes) during which treadmill exercise is performed. In the figure, “*” indicates that the p-value of the 300 mg/kg orotic acid administration group is less than 5% with respect to the vehicle administration group.

FIG. 6 shows transition of a heart rate during and after exercise in the vehicle administration group and the 300 mg/kg orotic acid administration group. In the figure, “Exercise” indicates the time (minutes) during which treadmill exercise is performed. In the figure, “*” indicates that the p-value of the 300 mg/kg orotic acid administration group is less than 5% with respect to the vehicle administration group.

FIG. 7 shows transition of a heat production amount during and after exercise in the vehicle administration group and the 300 mg/kg orotic acid administration group. In the figure, “Exercise” indicates the time (minutes) during which treadmill exercise is performed. In the figure, “*” indicates that the p-value of the 300 mg/kg orotic acid administration group is less than 5% with respect to the vehicle administration group.

FIG. 8 shows exercise economy in the vehicle administration group and the 300 mg/kg orotic acid administration group. In the figure, “**” indicates that the p-value of the 300 mg/kg orotic acid administration group is less than 1% with respect to the vehicle administration group.

### DESCRIPTION OF EMBODIMENTS

### [Body Temperature Lowering Agent and Body Temperature Elevation Suppressor]

A body temperature lowering agent according to an embodiment of the present invention contains at least one substance selected from the group consisting of orotic acid, a derivative thereof, and a salt of the orotic acid or the derivative (hereinafter, these may be collectively referred to as “orotic acids”) as an active ingredient.

A body temperature elevation suppressor according to an embodiment of the present invention contains at least one substance selected from the group consisting of orotic acids as an active ingredient.

In the present invention, orotic acid is also referred to as uracil-6-carboxylic acid. The orotic acid in the present invention includes both an orotic acid free form and an orotic acid hydrate, but the free form of orotic acid is preferred.

In the present invention, the derivative of orotic acid is not particularly limited as long as the derivative can be derived from orotic acid and has a body temperature lowering effect and a body temperature elevation suppressing effect. Examples thereof include dihydroorotic acid, orotidylic acid, orotidine, uracil, uridine, uridine monophosphate (UMP), uridine diphosphate (UDP), uridine triphosphate (UTP), deoxyuridine, deoxyuridine monophosphate (dUMP), deoxyuridine diphosphate (dUDP), deoxyuridine triphosphate (dUTP), thymine, 5-methyluridine, 5-methyluridine monophosphate (TMP), 5-methyluridine diphosphate (TDP), 5-methyluridine triphosphate (TTP), thymidine, thymidine monophosphate (dTMP), thymidine diphosphate (dTDP), thymidine triphosphate (dTTP), cytosine, cytidine, cytidine monophosphate (CMP), cytidine diphosphate (CDP), cytidine triphosphate (CTP), deoxycytidine, deoxycytidine monophosphate (dCMP), deoxycytidine diphosphate (dCDP), and deoxycytidine triphosphate (dCTP). Uridine monophosphate (UMP), uracil, uridine, cytidine, or the like, which is metabolized from orotic acid in a living body, is preferred. Note that RNA or DNA containing ribonucleotide or deoxyribonucleotide containing pyrimidine as described above can also be used as a source of the derivative of orotic acid in the present invention.

In the present invention, as salts in the salt of orotic acid or the salt of the orotic acid derivative, a salt acceptable as a food or a drug is preferred. Examples thereof include: alkali metal salts such as sodium salts and potassium salts; alkaline earth metal salts such as calcium salts and magnesium salts; heavy metal salts such as zinc salts; ammonium salts; basic amino acid salts such as arginine; amine salts such as triethylamine salts; choline salts; carnitine salts; and creatine salts.

Orotic acids used in the present invention may be commercially available products. Alternatively, orotic acids obtained as follows may be used: cultures are produced and accumulated in a culture solution by a fermentation method using various microorganisms in which a mutation of pyrimidine auxotrophy or pyrimidine analog resistance is introduced (see, for example, JP 2927882 B), and orotic acids are purified and collected from the above-described cultures or orotic acid-containing whey by usual purification means, for example, a separation purification method such as a precipitation method or a chromatography method using an ion exchange resin or active carbon.

In the present invention, the body temperature may be either the skin temperature, which is the temperature of the body surface, or the core body temperature, which is the temperature inside the body such as the brain or internal organs. The body temperature lowering agent according to an embodiment of the present invention can lower the core body temperature, which is an accurate body temperature not affected by the external environment. Meanwhile, the body temperature elevation suppressor according to an embodiment of the present invention can suppress elevation of the core body temperature.

The body temperature lowering agent according to an embodiment of the present invention lowers the body temperature at rest. The body temperature lowering agent according to the embodiment of the present invention can lower the body temperature at rest, and can be thus used for therapy (hypothermic therapy) for actively lowering the core body temperature to 32 to 34° C. for a patient with a severe brain injury disease (head injury, cerebrovascular disorder, after cardiac arrest syndrome, etc.), for example. The hypothermic therapy is considered to be a treatment that minimizes secondary brain damage because cerebroprotection is possible by reducing cerebral metabolism. For example, a hypothermic action at rest can protect nerves after a stroke episode. In general, a decrease in brain temperature of 1° C. results in a decrease in cerebral metabolism of about 7%, and the hypothermic therapy for after cardiac arrest syndrome is recognized worldwide and has become a standard treatment.

In addition, the body temperature lowering agent according to the embodiment of the present invention lowers the body temperature at rest but can maintain a healthy state for the body. Thus, the body temperature lowering agent can be used, for example, in a case where it is necessary to suppress body temperature elevation while maintaining a healthy state. Furthermore, it is expected that the body temperature lowering agent can be applied to hibernation and extension of life in the future.

The body temperature lowering action of the body temperature lowering agent according to the embodiment of the present invention can be measured by a method for measuring the core body temperature at rest. The core body temperature at rest can be measured in accordance with a known method, for example, the method described in an article of Takahashi et al. (Nature 583: 109-114, 2020) and the like. Specifically, the measurement can be performed in the following manner.

The core body temperature at rest is measured using a TRM54P Rat Telemetry System (available from KAHA Sciences) and SmartPad (TR181: available from KAHA Sciences) and quantified. The main body of TRM54P Rat Telemetry System is indwelled in the abdominal cavity of a test animal for measurement. All experiments are performed in the light phase, and on the day of the experiments, a rat is fasted 2 hours before start of the test to eliminate the effect of food-induced heat production. After the core body temperature at rest is measured for 20 minutes, a drug (orotic acids) or vehicle is intraperitoneally administered, and the core body temperature is measured for 120 minutes.

In a case where the core body temperature at rest is lowered as compared with a case where orotic acids are not administered, it can be determined that the orotic acids have a body temperature lowering action.

The body temperature elevation suppressor according to an embodiment of the present invention lowers body temperature during exercise. The body temperature elevation suppressor according to the embodiment of the present invention can lower the body temperature during exercise. Thus, for example, it is possible to suppress body temperature elevation during long-term endurance exercise such as marathon and to improve the long-term endurance exercise capacity. In addition, the body temperature elevation suppressor according to the embodiment of the present invention can also suppress excessive body temperature elevation. Thus, the body temperature elevation suppressor can be used for prevention and treatment of diseases caused by excessive body temperature elevation, such as heatstroke.

The body temperature elevation suppressing action of the body temperature elevation suppressor according to the embodiment of the present invention can be measured by a method for measuring the core body temperature during exercise. The core body temperature during exercise can be measured in accordance with a known method, for example, a method described in the article by Takahashi et al. (Nature 583: 109-114, 2020) and the like. Specifically, the measurement can be performed in the following manner.

A rat is placed in a treadmill metabolism chamber for small animals having an inclination of 10°, and after measuring the core body temperature at rest for 20 minutes, a single dose of a drug (orotic acids) is administered intraperitoneally. After the administration, the rat is left in a rest state for 15 minutes, and then exercise is started. The exercise is started at a running speed of 5 m/min, the running speed is increased to 20 m/min over 5 minutes, and the core body temperature is measured in the same manner as the method for measuring the core body temperature at rest.

In a case where the core body temperature elevation during exercise is suppressed as compared with a case where the orotic acids are not administered, it can be determined that the orotic acids have the body temperature elevation suppressing action.

The body temperature lowering agent and the body temperature elevation suppressor according to an embodiment of the present invention may contain only orotic acids, but may contain various compounding ingredients for pharmaceutical preparations such as pharmaceutically acceptable usual carriers, antioxidants, binders, stabilizers, excipients, diluents, pH buffers, disintegrants, solubilizers, solubilizing agents, isotonic agents, or the like. The dosage form of the body temperature lowering agent and the body temperature elevation suppressor according to an embodiment of the present invention may be a solid preparation such as a powder, a granule, or a capsule, or a liquid preparation such as a solution, an emulsion, or a suspension.

A method for administering the body temperature lowering agent according to an embodiment of the present invention is not particularly limited as long as the effect of lowering the core body temperature at rest is obtained, and may be oral administration or parenteral administration, but oral administration is preferable. Suitable examples of the parenteral administration method include enteral administration and intravascular administration (preferably intravenous administration). The dosage of the body temperature lowering agent according to an embodiment of the present invention may vary depending on symptoms, dosage forms, body weight, and the like. In a case of oral administration, the dosage may be 50 mg to 10 g, preferably 500 mg to 3 g per day for an adult (having a body weight of about 60 kg) in terms of orotic acids.

A method for administering the body temperature elevation suppressor according to an embodiment of the present invention is not particularly limited as long as the effect of suppressing elevation of the core body temperature during exercise is obtained, and may be oral administration or parenteral administration, but oral administration is preferable. Suitable examples of the parenteral administration method include enteral administration and intravascular administration (preferably intravenous administration). The dosage of the body temperature elevation suppressor according to an embodiment of the present invention may vary depending on symptoms, dosage forms, body weight, and the like, but in a case of oral administration, the dosage may be 50 mg to 10 g, preferably 500 mg to 3 g per day for an adult (having a body weight of about 60 kg) in terms of orotic acids.

The body temperature lowering agent and the body temperature elevation suppressor according to an embodiment of the present invention can be administered to vertebrates. Suitable examples of the vertebrates include animals belonging to mammals and animals belonging to birds. Among them, suitable examples include humans, monkeys, mice, rats, hamsters, guinea pigs, cows, pigs, horses, rabbits, sheep, goats, cats, dogs, chickens, and quails, and among them, more suitable examples include humans, livestock, and poultry.

### [Food Composition for Lowering Body Temperature and Food Composition for Suppressing Body Temperature Elevation]

Orotic acids have a body temperature lowering action, and can be thus contained in various food compositions as an active ingredient of a food composition for lowering body temperature in a case of lowering the body temperature at rest. In the present invention, the food composition includes not only a food but also a beverage.

In addition, orotic acids have a body temperature elevation suppressing action, and can be thus contained in various food compositions as an active ingredient of a food composition for suppressing body temperature elevation, for the purpose of preventing, treating, or improving a disease or a symptom caused by body temperature elevation such as body temperature elevation during exercise, heatstroke, or the like. In the present invention, the food composition includes not only a food but also a beverage.

Examples of the food composition for lowering body temperature and the food composition for suppressing body temperature elevation according to an embodiment of the present invention (hereinafter, collectively referred to as the food composition of the present invention) include various beverages, various dairy products such as yogurts, cheeses, butters, and lactic acid bacteria fermented products, liquid foods, jellies, candies, retort foods, tablet confections, cookies, sponge cakes, breads, and biscuits. The food composition of the present invention can also be used as a health food, a food with functional claims, a food for special dietary uses, a food for patients, a nutritional supplement, a supplement, a food for specified health uses, or the like.

In the food composition of the present invention, edible carbohydrates, proteins, lipids, vitamins, minerals, sugars (glucose and the like), natural or artificial sweeteners, citric acid, carbonated water, fruit juice, stabilizers, preservatives, binders, thickeners, emulsifiers, or the like can be appropriately blended.

The blending amount of the orotic acids in the food composition of the present invention is appropriately selected depending on the type of the food composition, an effect expected by administration of the food composition, and the like. The total amount of the active ingredients is 0.0001 to 100 mass %, preferably 0.001 to 100 mass %, more preferably 0.01 to 100 mass %.

In the food composition of the present invention, an amount of orotic acids generally ingested by an adult (having a body weight of about 60 kg) per day can be from 1 mg to 10 g, preferably from 10 mg to 5 g, more preferably from 50 mg to 1 g. The number of times of ingestion is not particularly limited, but is preferably 1 to 3 times per day. The number of times of ingestion may be increased or decreased as necessary. Note that the term “per day” refers to an indicated daily adequate intake, or an amount contained in one serving in a case of a drinkable beverage which is usually consumed at one time, although varying depending on the form of the food composition of the present invention.

The food composition described above may be a food composition having an indication that the food composition has a body temperature lowering action and/or a body temperature elevation suppressing action. Examples of the food composition having an indication that the food composition has a body temperature elevation suppressing action include food compositions having an indication that the food composition has a body temperature elevation suppressing action during exercise, an indication that the food composition has a preventing and/or treating a disease caused by body temperature elevation such as heatstroke, and the like.

### EXAMPLES

Hereinafter, the present invention will be described in detail with reference to examples, but the present invention is not limited to these examples.

### [Example 1] (Effect of Orotic Acid on Core Body Temperature at Rest)

A 10-week-old male Wistar rat was used, and a one-week preliminary breeding period was provided before the day of an experiment. During the experiment, room temperature was maintained at 22±2° C.

After the preliminary breeding, a telemetry (TRM54P, available from KAHA Sciences) for measuring the body temperature was indwelled in the abdominal cavity, and a recovery period and an acclimation period were provided for one week each. 300 mg/kg orotic acid, 30 mg/kg orotic acid, or a vehicle (1% carboxymethyl cellulose) was intraperitoneally administered to examine the effect on the core body temperature at rest. In addition to the core body temperature and a heart rate, an oxygen/carbon dioxide intake was measured using a breath gas analyzer (trade name: Oxymax, available from Columbus Instruments) to calculate a respiratory exchange ratio (also referred to as RER) and an amount of heat production. In addition, the amount of spontaneous activity was measured using an infrared beam sensor (trade name: beam sensor type spontaneous activity amount measuring device, available from Panlab).

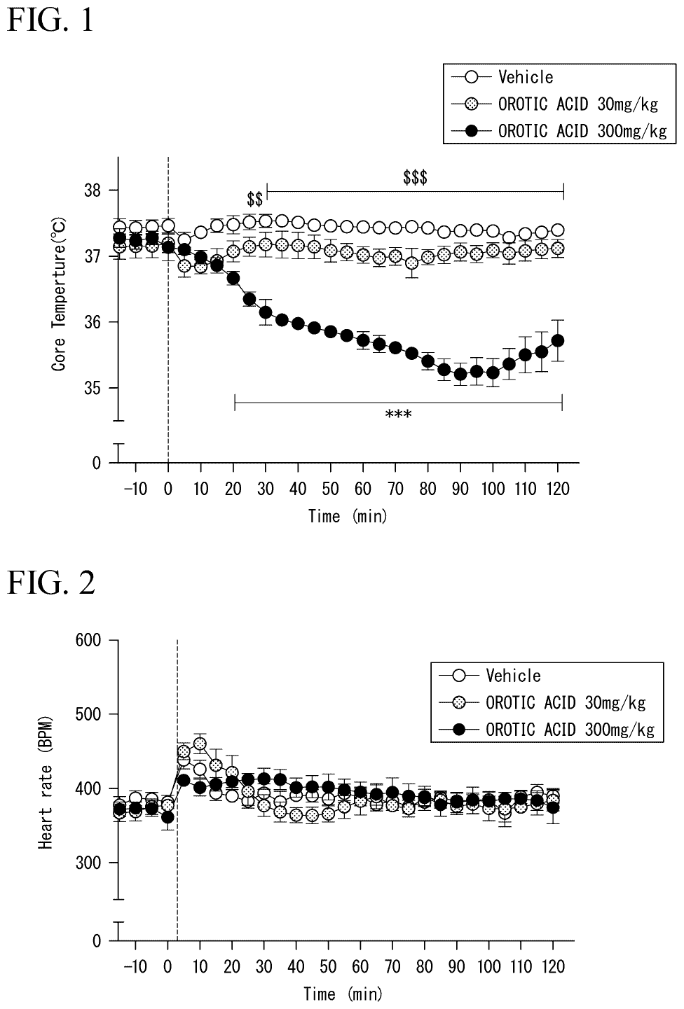

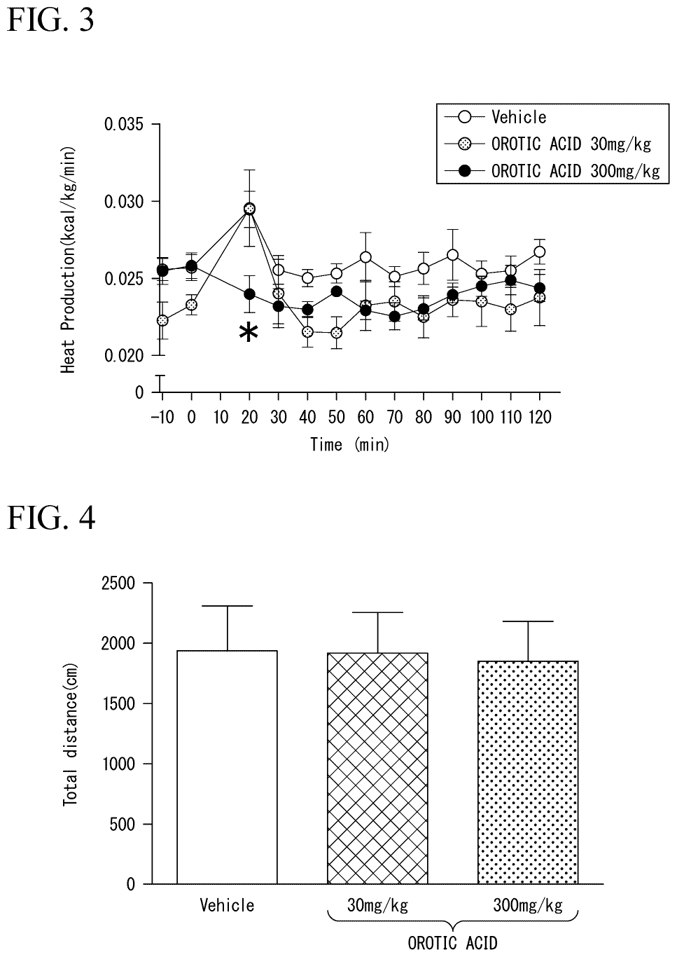

Note that in the experiment, the rat was placed under fasting for 2 hours before the start of the experiment, and the core body temperature at rest was measured for 20 minutes. Thereafter, the orotic acid or vehicle was intraperitoneally administered, and the measurement was performed until 2 hours later. The orotic acid was used in a free form. FIG. 1 shows transition of the core body temperature, FIG. 2 shows transition of the heart rate, and FIG. 3 shows transition of the amount of heat production. FIG. 4 shows the amount of spontaneous activity.

As shown in FIGS. 1 and 3, it was revealed that the core body temperature and the amount of heat production were significantly decreased only in the 300 mg orotic acid administration group and the core body temperature was decreased in a concentration dependent manner. There was no difference between the groups in the room temperature and the amount of spontaneous activity at this time, and thus, no factor affecting the core body temperature other than the administration of orotic acid was recognized.

On the other hand, a contraction phase, a diastole phase, and a mean blood pressure (not shown) were significantly increased in the orotic acid administered groups, and the heart rate did not fluctuate as shown in FIG. 2. Lowering of the core body temperature in rats is caused by an increase in heat dissipation due to suppression of heat production mainly based on metabolism and peripheral vasodilation. In a case where the heat dissipation is increased, the blood pressure is lowered and the amount of heat production measured by the breath gas analyzer is lowered. Thus, it is considered that the suppression of heat production further contributes to the effect of lowering the core body temperature by orotic acid.

The heart rate at rest is also used as an index of the amount of activity or the amount of heat production. From the value of the heart rate, it was also shown that the lowering of the core body temperature by orotic acid was an activity amount-independent body temperature lowering effect.

From the results of the present Example, it was revealed that orotic acid has an effect of suppressing heat production to lower the core body temperature.

### [Example 2] (Effect of Orotic Acid on Core Body Temperature During Exercise)

A 10-week-old male Wistar rat was used, and a one-week preliminary breeding period was provided before the day of an experiment. During the experiment, room temperature was maintained at 22±2° C.

After the preliminary breeding, a telemetry (trade name: TRM54P, available from KAHA Sciences) for measuring the body temperature was indwelled in the abdominal cavity, and a running test was performed after a recovery period of one week.

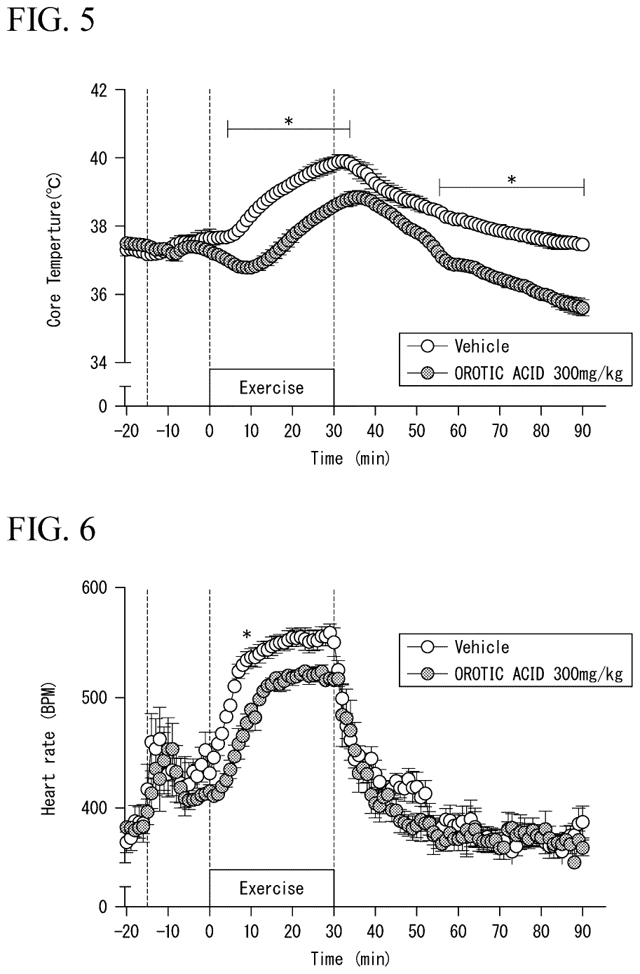

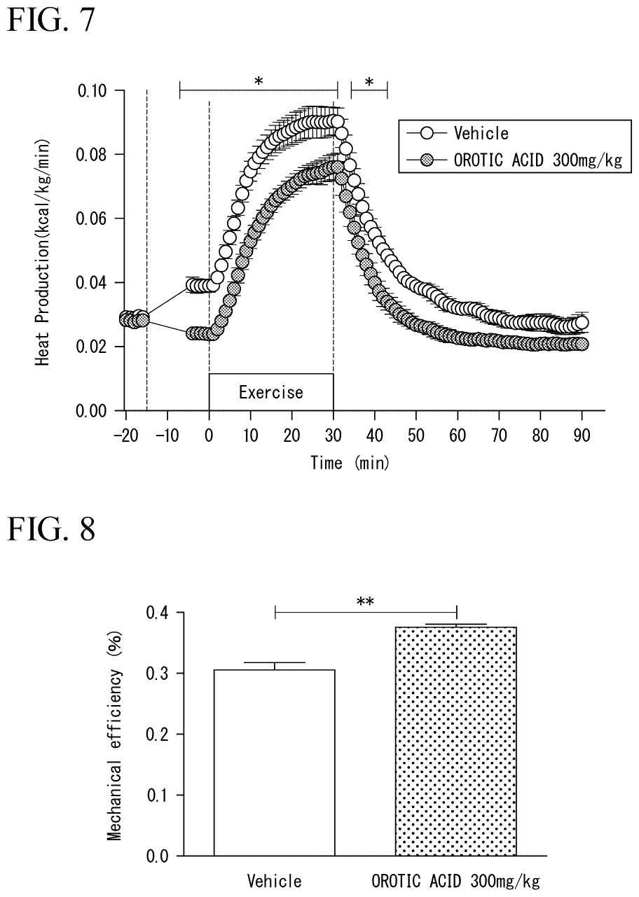

Note that in the experiment, after 300 mg/kg orotic acid or a vehicle (physiological saline) was intraperitoneally administered in a rest state, treadmill exercise (20 m/min) was performed for 30 minutes, and the measurement was performed until 1 hour after the end of the exercise. The measurement items were core body temperature, a blood pressure, a heart rate, and breath gas metabolism. From the obtained oxygen/carbon dioxide intake, exercise economy was calculated. The orotic acid was used in a free form. FIG. 5 shows transition of the core body temperature, FIG. 6 shows transition of the heart rate, and FIG. 7 shows transition of the amount of heat production. FIG. 8 shows the exercise economy.

As shown in FIG. 5, FIG. 7, and FIG. 8, intraperitoneal administration of 300 mg/kg orotic acid increased the exercise economy, lowered the amount of heat production, and suppressed excessive core body temperature elevation during exercise. From this result, it was found that orotic acid has a possibility of suppressing core body temperature elevation and extending the time for the core body temperature to reach 40° C. to improve endurance performance. In fact, as shown in FIG. 6, the heart rate during exercise, which is an index of relative intensity in endurance exercise, was significantly decreased, and thus it was found that there is a possibility that the orotic acid administration group has an effect of relatively easing exercise as compared with the vehicle group.

From the results of the present Example, it was revealed that orotic acid has an effect of enhancing exercise economy and suppressing core body temperature elevation during exercise.

### INDUSTRIAL APPLICABILITY

The body temperature lowering agent, the food composition for lowering body temperature, the body temperature elevation suppressor, and the food composition for suppressing body temperature elevation according to the present invention, which contain at least one substance selected from the group consisting of orotic acid, a derivative thereof, and a salt of the orotic acid or the derivative as an active ingredient, can lower the body temperature spontaneously and continuously and suppress excessive body temperature elevation. Thus, they can be used in emergency medical sites requiring brain cooling, suppression of body temperature elevation during long-term endurance exercise, and prevention and treatment of diseases caused by excessively high body temperature such as heatstroke.

1. A body temperature lowering agent comprising at least one substance selected from the group consisting of orotic acid, a derivative thereof, and a salt of the orotic acid or the derivative as an active ingredient.

2. The body temperature lowering agent according to claim 1, wherein the body temperature is core body temperature.

3. The body temperature lowering agent according to claim 1, wherein the orotic acid is a free form of orotic acid.

4. A body temperature elevation suppressor comprising at least one substance selected from the group consisting of orotic acid, a derivative thereof, and a salt of the orotic acid or the derivative as an active ingredient.

5. The body temperature elevation suppressor according to claim 4, wherein the body temperature is core body temperature.

6. The body temperature elevation suppressor according to claim 4, wherein the orotic acid is a free form of orotic acid.

7. A food composition for lowering body temperature, comprising at least one substance selected from the group consisting of orotic acid, a derivative thereof, and a salt of the orotic acid or the derivative as an active ingredient.

8. A food composition for suppressing body temperature elevation, comprising at least one substance selected from the group consisting of orotic acid, a derivative thereof, and a salt of the orotic acid or the derivative as an active ingredient.

9. The food composition according to claim 7, wherein the body temperature is core body temperature.

10. The food composition according to claim 7, wherein the orotic acid is a free form of orotic acid.

11. The food composition according to claim 8, wherein the body temperature is core body temperature.

12. The food composition according to claim 8, wherein the orotic acid is a free form of orotic acid.
